(19) United States
(12) Patent Application Publication
(10) Pub. No.: US 2024/0345094 A1
(43) Pub. Date: Oct. 17, 2024

(54) DOUBLE FRAGMENTATION ISOBARIC TAGGING

(71) Applicant: Saint Louis University, St. Louis, MO (US)

(72) Inventors: James Edwards, University City, MO (US); Christopher K. Arnatt, Manchester, MO (US); Michael Armbruster, Ballwin, MO (US)

(73) Assignee: Saint Louis University, St. Louis, MO (US)

(21) Appl. No.: 18/592,661

(22) Filed: Mar. 1, 2024

Related U.S. Application Data
(60) Provisional application No. 63/488,056, filed on Mar. 2, 2023.

(51) Int. Cl. G01N 33/58; G01N 30/02; G01N 30/72

(52) U.S. Cl. CPC G01N 33/58; G01N 30/7233; G01N 2030/027; G01N 2458/15

(57) ABSTRACT

The present disclosure provides constant-mass isobaric tags and methods for their use in multiplexed, double fragmentation mass spectrometry. The tags can be used for various aldehyde-, thiol-, amine-, and acid-containing analytes and can be used to quantify their relative amounts in different samples. Up to 192 samples can be combined for analysis.

### CROSS REFERENCE TO RELATED APPLICATIONS

This application claims the benefit of U.S. Provisional Patent Application No. 63/488,056, filed Mar. 2, 2023, the entire contents of which are incorporated by reference herein.

### STATEMENT REGARDING FEDERALLY SPONSORED RESEARCH OR DEVELOPMENT

This invention was made with government support under awarded by NIH-NIGMS (5R01GM134081). The government has certain rights in the invention.

### FIELD OF INVENTION

The present disclosure provides constant-mass isobaric tags and methods for their use in multiplexed, double fragmentation mass spectrometry. The tags can be used for various aldehyde-, thiol-, amine-, and acid-containing analytes and can be used to quantify their relative amounts in different samples.

### BACKGROUND OF INVENTION

Liquid chromatography-mass spectrometry (LC-MS) analysis of biological thiols suffers from poor analyte stability and quantitation. Derivatization with chemical tags can improve signal response with the addition of a hydrophobic and proton affinity moiety while stabilizing the sulfhydryl group. These tagging reactions are often performed using iodoacetamide (IAM), maleimide, or one of their derivatives. Stabilizing the sulfhydryl group also allows for accurate discrimination between reduced and oxidized thiols in cells. Despite their prevalence in proteomics, adoption and development of thiol derivatization methods has been limited in metabolomics with examples including α-iodoacetamide QDE, bromoacetonylquinolinium bromide, iodoTMT, and cICAT. These methods improve quantitation and chromatographic performance while incorporating isotopes to increase throughput.

Isotope incorporation onto chemical tags allows for simultaneous LC-MS analysis of multiple samples. This reduces instrument drift and provides equal ionization for each analyte across all isotope variants. Isobaric labeling specifically is used to multiplex without increasing MS¹ spectral complexity. In this case, all tag variants contain the same number, but different placement of isotopes. Isobaric tags with fixed-mass reporters (TMT, iTRAQ, DiART, DiLeu) often rely on al type fragments with a tertiary amine due to their efficient reporter formation. While this structural motif provides quantitative reporters, the number of modifiable balancer isotopes is limited without adding additional mass to the tag. These constant-mass reporters can also suffer from reporter distortion due to co-isolation.

Neutral loss-based isobaric tags present a solution to chimeric spectra acquired during MS² scans. The reporter group stays attached to the analyte and provides a unique mass shift for each analyte in the MS² scan. This allows the MS² mass analyzer to differentiate between co-isolated precursors by the mass difference of the reporter. This type of neutral loss tagging was recently applied using a quaternary amine tag to quantitate tagged acid metabolites. A trimethylamine neutral loss produced a cyclized reporter which was still attached to the analyte. This fragmentation regime has been explored in detail for both trimethylamine and dimethylsulfonium tags. The unique structure of these neutral loss tags provides many points of isotope modification on the balancer group with cost-efficient reagents like formaldehyde and methyl iodide. Despite their structural advantages, neutral loss tags have not reached the level of multiplexing of traditional, constant-mass reporter tags. One possible explanation for this discrepancy is their high mass reporters which limit the viability of neutron encoding due to the resolution drop-off on orbitrap systems.

### SUMMARY OF INVENTION

The present disclosure provides an isobaric, double fragmentation mass spectrometry tag, wherein the tag is selected from the group consisting of:
a thiol tag of structure

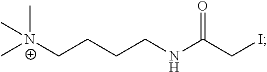

an amine tag of structure

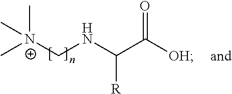

an acid and aldehyde tag of structure

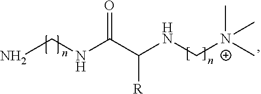

wherein R is H or unsubstituted alkyl; wherein n is 3 to 6; and wherein the tag has at least one N replaced by ¹⁵N, at least one C replaced by ¹³C, or at least one H replaced by deuterium (D).

The present disclosure is further directed to a method of multiplexed mass spectrometry for quantifying one or more analytes of interest in two or more samples, wherein the method comprises the following steps:
A) each sample is tagged with a different tag as described elsewhere in this disclosure, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight;
B) the samples are combined and processed via liquid chromatography;
C) a low resolution MS¹ scan is performed to identify the one or more analytes of interest and the tagged one or more analytes are fragmented to cyclize; and
D) secondary fragments of the tags are produced after a second fragmentation event and a high resolution MS² scan is performed to quantify the relative amount of the one or more analytes in each sample.

A further aspect of the present disclosure is a kit for tagging two or more samples for multiplexed mass spectrometry of one or more analytes of interest, the kit comprising at least two of the isobaric, double fragmentation mass spectrometry tags as described elsewhere in this disclosure, wherein the kit comprises one tag for each sample, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts isotope incorporation onto the 6-plex isobaric tags. Each tag has a total of 12 isotopes to produce a nominal m/z of 311 and are named based on the number of deuterium, ¹³C, and ¹⁵N isotopes.

FIG. 1B depicts HRMS¹ spectra of each 6-plex tag injected individually. Each synthesized tag was direct injected and characterized by HRMS at a resolution of R=140K.

FIG. 2A depicts tagged thiols fragment structures used to track formation efficiency of the cyclized product and constant mass reporters

FIG. 2B depicts optimal fragmentation energies determined by collision energy ramps.

FIG. 3A depicts two isobaric tags showing different mass reporters despite their same MS¹ nominal m/z.

FIG. 3B depicts mass limits imposed by orbitrap resolution decay for neutron encoded peaks spaced 2.9 mDa apart.

FIG. 3C depicts example spectra of 1:1 mixed 6-plex injection of N-acetylcysteine. Spectral overlap of reporters is avoided by high resolution MS² scans, shown by the inset spectra for m/z 150 and 152.

FIG. 3D depicts reaction pH optimization of thiol metabolites. Thiol standards were reacted for 16 hours in varying pH ammonium bicarbonate solutions (n=3). Optimal reaction efficiency was achieved at pH 9 without the overlabeling that can occur at elevated pH.

FIG. 4A depicts MS¹ extracted ion chromatograms (XICs) of the capillary HILIC separation of 6-plex tagged thiols.

FIG. 4B depicts MS² XICs of the 6 reporters showing co-elution across all tags despite differential deuterium labeling.

FIG. 5A depicts cell treatment and sample prep. Six total samples were treated, tagged, and mixed for analysis in a single injection. The MS¹ mass spectra is shown for glutathione, with the MS² spectra below zoomed to show 6 reporters.

FIG. 5B depicts changes in thiol metabolism upon γ-glutamyl-cysteine synthetase inhibition. * p<0.05, ** p<0.01.

FIG. 6 depicts the general synthetic route to produce dual fragmentation tags. Quaternary amine tags are coupled to alpha-keto acids through a reductive amination. This structure can undergo further modification to the acid group to expand the classes of targeted metabolites.

FIG. 7A depicts tagging and fragmentation scheme for the analysis of amine containing metabolites. The quaternary amine tag contains sites for isotope incorporation to the balancer or reporter group. The amine tag is activated with 4-(4,6-Dimethoxy-1,3,5-triazin-2-yl)-4-methylmorpholinium chloride (DMTMM) in dimethylformamide (DMF) to create an amine reactive tag. Once activated, the tag is added to the biological sample to react for 2 hours, creating tagged metabolites. Additional reactive groups may also be created from this reactive acid group: amine (through diamine coupling), iodoacetamide, boronate, maleimide, alkene, isothiocyanate, and sulfonyl chloride. This tag undergoes fragmentation at the bond indicated by a dashed line to cyclize.

FIG. 7B depicts how experimental data has validated this fragmentation route for R=H and CH₃, and this pattern is expected to hold for longer alkyl chains. The tag cyclizes and then undergoes further fragmentation to liberate a reporter ion which can be used for quantitation. Dashed lines indicate sites of bond breakage upon collision induced dissociation (CID).

FIG. 8 depicts how attachment of the dual fragmentation tag (with R=H) to the amine moiety of amino acids through DMTMM coupling produced the reporter with exceptional efficiency. The cyclized intermediate is also observed at a lower intensity. The quaternary amine tag here contained a CD₃ group which is lost during fragmentation as part of the balancer group.

FIG. 9 depicts how expansion of the R group to CH₃ allows the synthesis using pyruvate as a precursor by attaching the quaternary amine tag to the alpha-keto group using a reductive amination.

FIG. 10 depicts how the pyruvate variant of the quaternary amine tag produces heavier reporters (m/z 98 vs. 84) with more sites for isotope incorporation. Reporter generation is still efficient, while the cyclized intermediate is also observed.

FIG. 11 depicts experimental data using the pyruvate variant of the quaternary amine tag with n=3 for the chain length. This produces a 4 membered ring on the reporter at m/z 84.081.

FIG. 12 depicts the synthetic route to expand targeted metabolites. Attaching a mono-boc amine to the acid group on the tag through amide coupling followed by deprotection produces a primary amine reactive group which can be used to target acid metabolites.

FIG. 13A depicts how the acid-reactive tag contains two sites for balancer isotope incorporation. The carbon chain lengths of balancer 2 and the reporter region can differ but are expected to be within C=3-6. The tag can be reacted with acid metabolites through amide coupling mediated by DMTMM. Like the amine-targeting variant, this tag undergoes fragmentation at the bond indicated by a dashed blue line to cyclize.

FIG. 13B depicts the expected fragments from the tag of FIG. 13A. The cyclized fragmentation product undergoes further dissociation to produce the reporter ion. Dashed lines indicate sites of bond breakage upon collision induced dissociation (CID). n=4 was used for the reporter ion chain length here.

FIG. 14A depicts an example fragmentation spectrum for singly charged tagged benzoic acid. Both charge states fragment to produce the reporter ion at m/z 98. n=4 for both the balancer and reporter chain length in this example.

FIG. 14B depicts an example fragmentation spectrum for doubly charged tagged benzoic acid. Both charge states fragment to produce the reporter ion at m/z 98. n=4 for both the balancer and reporter chain length in this example.

FIG. 15 depicts an example fragmentation spectrum for tagged butyric acid. Here only one charge state is observed, and both the cyclized product (m/z 284) and the reporter ion (98) are generated with great efficiency.

### DETAILED DESCRIPTION OF INVENTION

This disclosure describes an isobaric, double fragmentation mass spectrometry tag, wherein the tag is selected from the group consisting of:
a thiol tag of structure

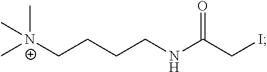

an amine tag of structure

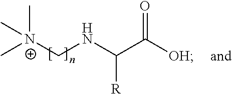

an acid and aldehyde tag of structure

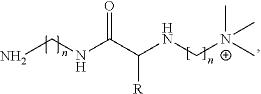

wherein R is H or unsubstituted alkyl; wherein n is 3 to 6; and wherein the tag has at least one N replaced by ¹⁵N, at least one C replaced by ¹³C, or at least one H replaced by deuterium (D).

The unsubstituted alkyl can be a C₁-C₃ linear or branched alkyl. In certain embodiments, R can be CH₃.

n can be 3, 4, 5, or 6. In certain embodiments, n is 4.

Any number of N, C, and H atoms can be replaced by ¹⁵N, ¹³C, and D, respectively. In certain embodiments, at least 12 total N, C, and H atoms are replaced by ¹⁵N, ¹³C, and D, respectively.

The tag can be selected from the group consisting of

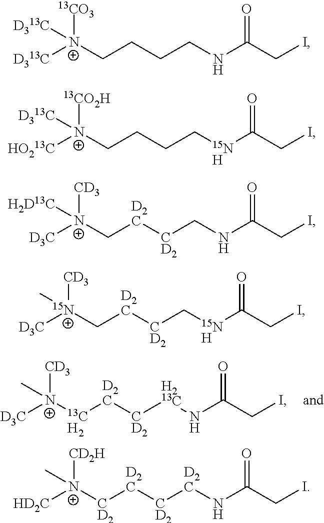

The present disclosure is further directed to a method of multiplexed mass spectrometry for quantifying one or more analytes of interest in two or more samples, wherein the method comprises the following steps:
A) each sample is tagged with a different tag as described elsewhere in this disclosure, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight;
B) the samples are combined and processed via liquid chromatography;
C) a low resolution MS¹ scan is performed to identify the one or more analytes of interest and the tagged one or more analytes are fragmented to cyclize; and
D) secondary fragments of the tags are produced after a second fragmentation event and a high resolution MS² scan is performed to quantify the relative amount of the one or more analytes in each sample.

In certain embodiments, each sample comprises lysed cells. In certain embodiments, each sample is derived from a bodily fluid or body tissue. The bodily fluid can be selected from the group consisting of whole blood, plasma, urine, lymph, cerebrospinal fluid, saliva, semen, synovial fluid, and peritoneal fluid. The body tissue can be epithelial tissue, muscle tissue, or nervous tissue. In certain embodiments, each sample is derived from a human or another mammal.

The method can be used for research or medical purposes. In certain embodiments, each sample corresponds to a different experimental condition. In certain embodiments, each sample is taken from one or more patients for the purpose of diagnosis or monitoring.

The method can have six samples or up to 192 samples.

Each tag can be selected from the group consisting of

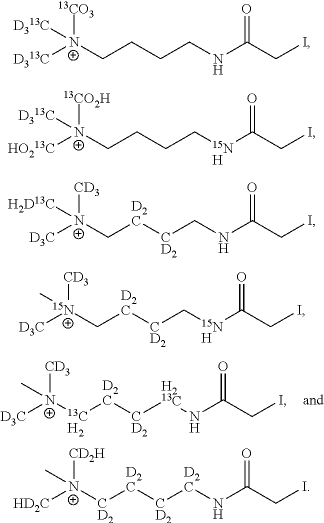

In certain embodiments, the one or more analytes can comprise thiol-containing metabolites related to GSH metabolism. The one or more analytes can be selected from the list consisting of Cys, Homocysteine, NAC, Cys-Gly, γ-Glu-Cys, GSH, and combinations thereof. In certain embodiments, the one or more analytes is one or more amino acids.

The liquid chromatography can be bare silica chromatography.

Another aspect of the disclosure is a kit for tagging two or more samples for multiplexed mass spectrometry of one or more analytes of interest, the kit comprising at least two of the isobaric, double fragmentation mass spectrometry tags described elsewhere in this disclosure, wherein the kit comprises one tag for each sample, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight.

The kit can comprise at least six tags or up to 192 tags. In certain embodiments, each tag is selected from the group consisting of

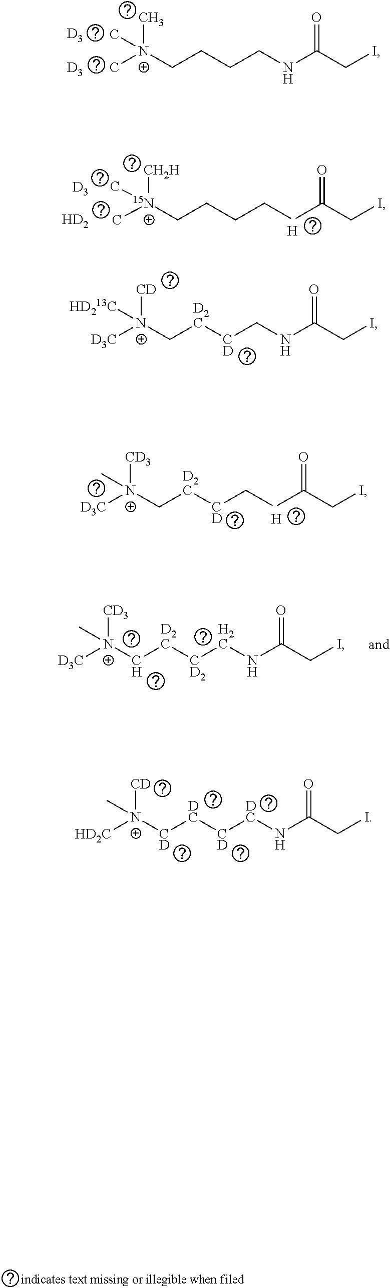

The kit can be used in the methods of multiplexed mass spectrometry described elsewhere in this disclosure. The kit can further include components for sample preparation and/or performing liquid chromatography and/or mass spectrometry. The kit can also further include an instruction manual for performing the method.

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

### Example 1: Materials and Methods

The following materials and methods are used throughout the rest of the examples.

### Standard Stock Creation:

Thiol metabolite standards and formic acid were purchased from Sigma Aldrich (St. Louis, MO). Iodoacetyl chloride, LC-MS grade water, acetonitrile (ACN), ammonium hydroxide, acetic acid, and ammonium acetate were purchased from Fisher Scientific (Pittsburgh, PA).

### Tag Synthesis:

Isotopic variants of 1,2-dibromoethane, potassium cyanide, formaldehyde, and methyl iodide were purchased from Cambridge Isotope Labs (Andover, MA, USA).

Synthesis of Tags with a Four Carbon Chain:

### General Procedure for Succinonitrile Isotopologue Synthesis:

The corresponding 1,2-dibromoethane starting material was dissolved in MeOH (or MeOD for d4-dibromoethane) in a microwave reaction vial. The corresponding potassium cyanide (KCN, K¹³CN, KC¹⁵N or K¹³C¹⁵N) was added (2 eq) to the solution. The suspension was heated to 140° C. and stirred for 20 minutes in a microwave synthesizer. The resulting solution was filtered to and washed with acetone (10 mL×3). The filtrate was concentrated to yield succinonitrile as a dark red waxy solid.

### General Procedure for Di-Tert-Butyl Butane-1,4-Diyldicarbamate Isotopologue Synthesis:

The corresponding succinonitrile isotopologue was dissolved in dry MeOH and cooled to 0° C. in a 1 L round bottom flask under nitrogen atmosphere. Di-tert-butyl decarbonate (2 eq) and NiCl₂ hexahydrate (20 mol %) were added to the stirring solution. NaBH₄ (14 eq) added in small portions over 30 minutes, adding ice as needed. The reaction mixture was allowed to warm to room temperature and stirred overnight. Diethylene triamine (8 eq) added and the solution was stirred for 30 minutes. The reaction mixture was concentrated, dissolved in 50 mL EtOAc, and washed with NaHCO₃ (50 mL). The aqueous layer was extracted with EtOAc twice more (50 mL×2). The combined organic layer was dried with MgSO₄ and concentrated to yield di-tert-butyl butane-1,4-diyldicarbamate as a yellow solid.

### Deprotection of Diboc-Protected Amines to Yield Putrescine Isotopologues:

The diboc-protected diamine intermediates were deprotected at RT in a stirred 4M HCl/Dioxane solution overnight. The solutions were concentrated and recrystallized in 9:1 EtOH/H₂O, filtered, and isolated as the HCl salt of the corresponding putrescine isotopologue.

### General Procedure for Tert-Butyl N-(4-Aminobutyl) Carbamate Isotopologue Synthesis:

1,4-diaminobutane (3 mmol) was dissolved in 10 mL EtOH and triethylamine (3 eq) was added. The reaction mixture was stirred while tert-butyl phenyl carbonate (3 mmol) was added slowly. The reaction mixture was heated to 160° C. and stirred for 20 minutes in a microwave synthesizer. The pH was adjusted to 3 with 2M HCl and washed with DCM (3×16 mL). The aqueous layer was basified to pH 10 with 2M NaOH and extracted with DCM (5×20 mL), dried with K₂CO₃, and concentrated to afford tert-butyl N-(4-aminobutyl) carbamate as a dark red oil.

General Procedure for Methylation of Tert-Butyl N-(4-Aminobutyl) Carbamate with Formaldehyde:

Tert-butyl N-(4-aminobutyl) carbamate was dissolved in 5 mL dry ACN. To the stirred solution, the corresponding isotopic variant of formaldehyde solution (20%) (3 eq) was added, followed by NaBH₃CN (2.6 eq). The reaction mixture was stirred for 15 minutes before adjusting the pH to 7 with acetic acid, several 3\acute{Å} molecular sieves were added, and the solution was stirred for an additional 2 hours. The reaction mixture was evaporated and reconstituted in 2M KOH (10 mL), extracted with ether (20 mL×3), and washed with 40 mL 0.5M KOH. The organic layer was dried with K₂CO₃ and concentrated to yield the dimethylated product, tert-butyl N-(4-N′,N′-dimethylaminobutyl) carbamate, as a colorless oil.

General Procedure for Methylation of Tert-Butyl N-(4-Aminobutyl) Carbamate with Methyl Iodide:

In a scalable microwave reaction vial, tert-butyl N-(4-aminobutyl) carbamate or tert-butyl N-(4-N′,N′-dimethylaminobutyl) carbamate was dissolved in 5 mL ACN and K₂CO₃ (4 eq) was added. The solution was stirred and methyl iodide (5 eq) was added dropwise to the stirred solution. The vial was heated to 130° C. and stirred for 20 minutes in a microwave reactor. The vial cap was then removed and two mL water was added to the vial. The top layer was extracted by pipette, dried and suspended in dichloromethane. The resulting solution was filtered by 0.45 μm syringe tip filter and concentrated to yield a dark red oil.

### Deprotection of Boc-Protected Amines to Yield Isotopic Tags:

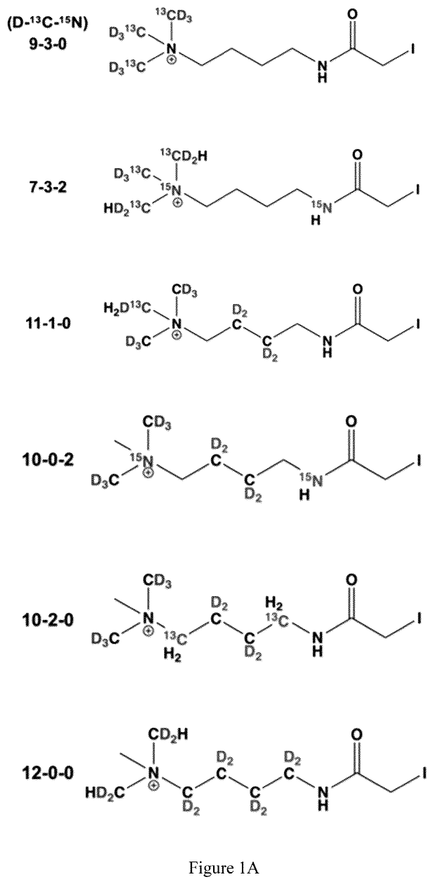

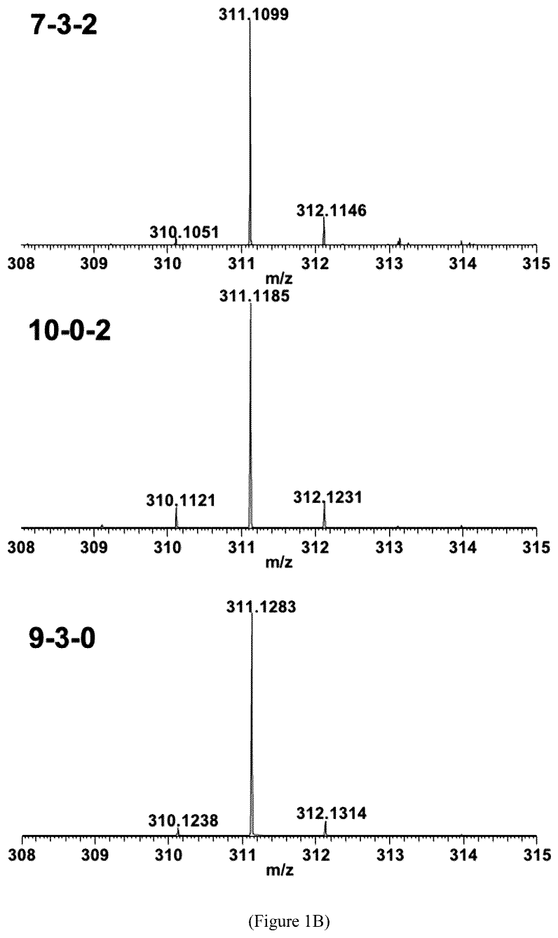

The boc-protected quaternary ammonium ion intermediates were deprotected at RT in a stirred 4M HCl/Dioxane solution overnight. The solutions were concentrated and purified via normal phase column chromatography (DCM/MeOH gradient over 10 minutes with an isocratic hold at 100% MeOH for 5 minutes) to yield the isotopic tags as HCl salts. All isotope labelled tags were characterized by high-resolution MS (FIG. 1A-1B).

Synthesis of Tags with a Three-Carbon Chain:

### General Procedure for 1,3-Diaminopropane Dihydrochloride Isotopologue Synthesis:

The corresponding 1,3-dibromopropane isotopologue was dissolved in 10 mL DMF. Depending on the desired isotopologue product, ¹⁴N or ¹⁵N Potassium phthalimide (2.2 eq) was added and the reaction mixture was heated and stirred for 3 hours at 80° C. Ice cold water was then added to form a white precipitate. The precipitate was collected by vacuum filtration and transferred to a microwave reaction flask. The white solid was suspended in 5 mL 1:1 concentrated HCl/Acetic acid solution and sealed. The reaction flask was heated to 170° C. and stirred for four hours in a microwave synthesizer. The mixture was concentrated in-vacuo and redissolved in DI water. To a chromatography column loaded with DOWEX 50W-X8 resin and primed with DI water, the dissolved mixture was added and eluted with an HCl gradient from 0.5M to 2M. The product was tracked by ninhydrin staining and those fractions were combined, concentrated, and recrystallized in 95% EtOH to yield 1,3-diaminopropane as a dihydrochloride salt.

### General Procedure for Tert-Butyl N-(3-Aminopropyl) Carbamate Isotopologue Synthesis:

1,3-diaminopropane dihydrochloride (3 mmol) was dissolved in 10 mL EtOH and triethylamine (3 eq) was added (for free amine starting materials, the triethylamine step was omitted). The reaction mixture was stirred while tert-butyl phenyl carbonate (3 mmol) was added slowly. The reaction mixture was heated to 160° C. and stirred for 20 minutes in a microwave synthesizer. The pH was adjusted to 3 with 2M HCl and washed with DCM (3×16 mL). The aqueous layer was basified to pH 10 with 2M NaOH and extracted with DCM (5×20 mL), dried with K₂CO₃, and concentrated to afford tert-butyl N-(3-aminopropyl) carbamate as a yellowish oil.

General Procedure for Methylation of Tert-Butyl N-(3-Aminopropyl) Carbamate with Formaldehyde:

Tert-butyl N-(3-aminopropyl) carbamate was dissolved in 5 mL dry ACN. To the stirred solution, the corresponding isotopic variant of formaldehyde solution (20%) (3 eq) was added, followed by NaBH₃CN (2.6 eq). The reaction mixture was stirred for 15 minutes before adjusting the pH to 7 with acetic acid, several 3 Å molecular sieves were added, and the solution was stirred for an additional 2 hours. The reaction mixture was evaporated and reconstituted in 2M KOH (10 mL), extracted with ether (20 mL×3), and washed with 40 mL 0.5M KOH. The organic layer was dried with K₂CO₃ and concentrated to yield the dimethylated product, tert-butyl N-(3-N′,N′-dimethylaminopropyl) carbamate, as a colorless oil.

General Procedure for Methylation of Tert-Butyl N-(3-Aminopropyl) Carbamate with Methyl Iodide:

In a scalable microwave reaction vial, tert-butyl N-(3-aminopropyl) carbamate or tert-butyl N-(3-N′,N′-dimethylaminopropyl) carbamate was dissolved in 5 mL ACN and K₂CO₃ (4 eq) was added. The solution was stirred and methyl iodide (5 eq) was added dropwise to the stirred solution. The vial was heated to 130° C. and stirred for 20 minutes in a microwave reactor. The vial cap was then removed and two mL water was added to the vial. The top layer was extracted by pipette, dried and suspended in dichloromethane. The resulting solution was filtered by 0.45 μm syringe tip filter and concentrated to yield a yellowish oil.

Deprotection of Boc-Protected Amines to Yield Isotopic Tags with a Three Carbon Chain:

The boc-protected quaternary ammonium ion intermediates were deprotected at RT in a stirred 4M HCl/Dioxane solution overnight. The solutions were concentrated and purified via normal phase column chromatography (DCM/MeOH gradient over 10 minutes with an isocratic hold at 100% MeOH for 5 minutes) to yield the isotopic tags as HCl salts. All isotope labelled tags were characterized by high-resolution MS.

Cell culture and lysis: Bovine aortic endothelial cells between passage number 7 and 10 were cultured to confluency in 6 cm plates with Dulbecco's modified eagle medium (DMEM) supplemented with 10% fetal bovine serum (FBS) at 37° C. and 5% CO₂. Cell media was swapped with DMEM supplemented with 2% FBS and 250 μM BSO. Control plates were swapped to DMEM with 2% FBS. All plates were lysed after 16 hours as previously described (Filla, L. A., et al., Anal Bioanal Chem 2019, 411 (24), 6399-6407). Briefly, the media was removed and the cells were rapidly rinsed with 1 mL of phosphate-buffered saline. Cells were lysed with the addition of 500 μL of cold 80/20 MeOH/H₂O containing 100 mM formic acid and 5 μM of D4 homocysteine as an internal standard. Plates were placed on a dry ice-ethanol bath and the cell contents were collected, then centrifuged at 21,100×g for three minutes. The supernatant was immediately collected and stored on dry ice until derivatization.

Thiol Derivatization: Thiol metabolite stocks were created as individual 500 μM vials with 50/50 H₂O/MeOH containing 20 mM formic acid and stored at −20° C. to minimize degradation. Lower concentration stocks were produced by further dilution and derivatized immediately. For cell lysate reactions, 100 μL of the lysate was used for each reaction. Thiol metabolites were added to 100 μL of 100 mM ammonium bicarbonate pH 8. An isotopic iodide tag was then immediately added (100 μL, 100 μM) and left at room temperature to react for 16 hours to ensure a complete reaction. All iodide tags and reactions were handled in the dark to prevent degradation. After the reaction was completed, the samples were mixed with the other isotope variants, dried in a vacuum centrifuge, and reconstituted to 100 μL in the starting mobile phase conditions.

Liquid Chromatography: Fused silica capillary (Polymicro Technologies, Phoenix, AZ) was used to fabricate columns with an inner diameter of 50 μm as previously described (Edwards, J. L., et al, Journal of Chromatography A 2007, 1172 (2), 127-134). Briefly, 50 μm inner diameter capillary was cut to 25 cm, a photopolymerized frit placed 30 mm from the end, then a tip pulled with a Sutter P2000 micropipette puller and etched in 50% HF. The capillary bed was packed to 20 cm with Atlantis silica HILIC 3 μm particles (Waters Corp, Milford, MA). After equilibration the capillary was cut to 17.5 cm for analysis. Flow was delivered by a Thermo Vanquish LC (Thermo Fisher Scientific, Waltham, MA) connected to a stainless steel tee to split the flow. A 50 μm×100 cm open capillary was used as the split to direct 115 nL of the total flow and 4 nL injections to the capillary column (split ratio 1:1500 for bulk flow of 175 μL/min and injection of 6 μL). The split ratio was determined by calculating flow through the capillary based on the experimental dead time and column dimensions. Mobile phase A was pure acetonitrile and mobile phase B was 25 mM ammonium acetate in 80/20 (water/ACN) with 0.05% acetic acid as an additive. The gradient is supplied in Table 1 below.

TABLE 1
Gradient used for HILIC separation of tagged thiol metabolites.

| Time (min) | % B |
|---|---|
| 0.0 | 40 |
| 0.5 | 40 |
| 10.0 | 90 |
| 12.0 | 90 |
| 12.1 | 40 |
| 20.0 | 40 |

MS Analysis: A Q-Exactive mass spectrometer (Thermo Fisher Scientific, Waltham, MA) was used for all analyses. An MS¹ survey scan operated at a resolution of 17.5K with an ion injection time of 50 ms and AGC of 1e⁶ for m/z 250-650. Two PRM scans followed each survey scan, alternating through an inclusion list of 7 analytes. These MS² scans operated at a resolution of 140K with an ion injection time of 200 ms, AGC of 1e⁵, and the optimized collision energy determined for each analyte. All runs were operated with a spray voltage of 1.75 kV and a capillary temperature of 200° C. Collision energy optimization was performed by direct infusion at 5 μL/min with each tagged thiol at 1 μM.

Data Analysis: LC-MS data extraction was performed in Skyline and QualBrowser. Further statistical analysis was performed using Graphpad Prism 9 and R version 4.1.2. Raw reporter intensities were first corrected by accounting for signal lost to isotopic impurities (Table 2), then normalized to the spiked internal standard D₄-homocysteine to account for differences in reaction efficiency and sample handling.

TABLE 2
Isotope purity of each isobaric tag.

| Tag | |
|---|---|
| (D-¹³C-¹⁵N) | Isotope purity (%) |
| 7-3-2 | 85.3% |
| 9-3-0 | 90.4% |
| 10-0-2 | 77.2% |
| 10-2-0 | 79.7% |
| 11-1-0 | 85.0% |
| 12-0-0 | 83.1% |

The isotope purity was assessed taking the peak intensity ratio of the expected isotope variant to the total peak intensity of all observed isotope variants.

### Example 2: Tag Design and Mass Spectrometry

Here is presented a six-plex isobaric, thiol tagging scheme which produces constant mass reporters using a double fragmentation approach. The tags leverage a sequential fragmentation where first a quaternary alkylammonium neutral loss produces a cyclized product. This product undergoes further fragmentation to liberate an isotope-encoded reporter with a constant mass similar to iodo-TMT. The cyclization described here has been used to produce neutral-loss based isobaric tags previously, but never for fixed-mass reporters. The quaternary alkylammonium allows for cost-efficient incorporation of many isotopes to the balancer group, up to +13 Da, and negates deuterium chromatographic shifts. The alkyl reporter region is synthesized using affordable potassium cyanide and dibromoethane isotopes with a maximum of +8 Da shown. The method was used to investigate GSH metabolism changes upon γ-glutamyl-cysteine synthetase inhibition across six samples in a single injection. Overall, this provides a method for creating constant-mass isobaric tags.

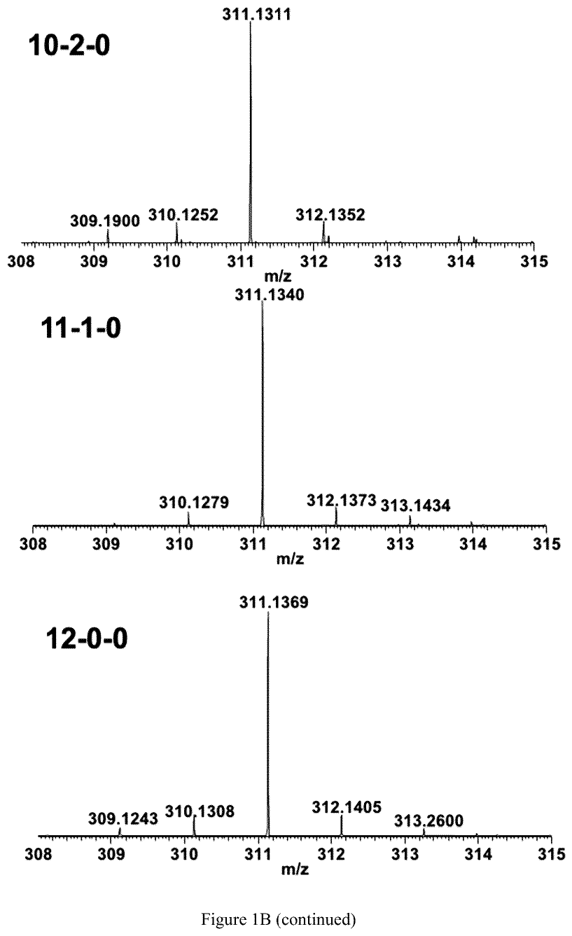

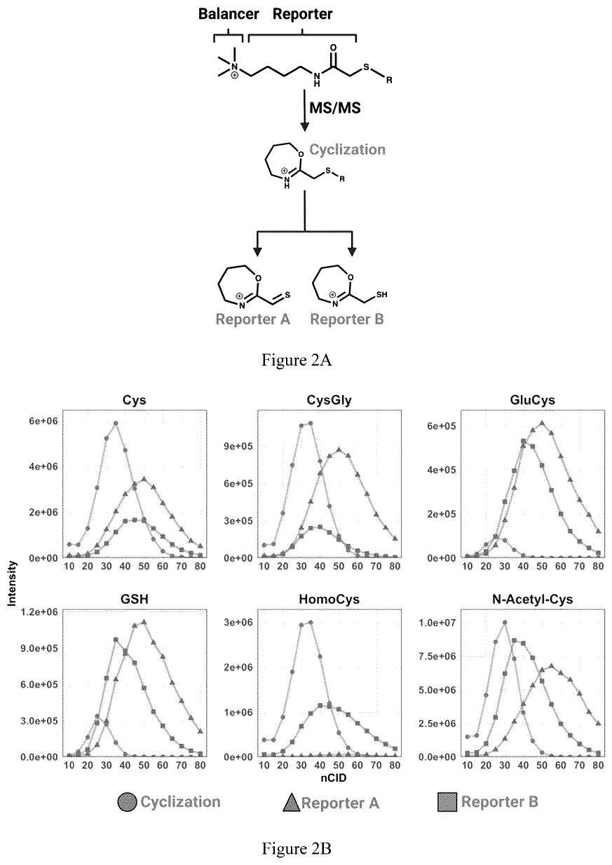

Six isobaric tags were synthesized by the addition of iodoacetyl chloride to a previously published tag structure which contain a primary amine and ²H, ¹³C, and ¹⁵N isotopes (Armbruster, M., et al., Anal Chim Acta 2022, 1190, 339260). These tags are denoted by the number of isotopes (D-¹³C-¹⁵N). The number and placement for these tags has been expanded from their first report to provide additional isotope modification on the alkyl chain. The exact mass of each tag spans a range of 27 mDa, allowing for simple isolation with a quadrupole prior to fragmentation (Fang, H., et al., Analytical Chemistry 2016, 88 (14), 7198-7205). This small mass difference is unresolved in the low resolution MS¹ scan which minimizes spectral complexity. Upon fragmentation the tagged analytes produce cyclized reporters (FIG. 2A) similar to those previous reported. A pair of additional fragments emerge at higher collision energies which are independent of the precursor mass (FIG. 2B). These reporters are referred to as reporter A and B for the lighter and +2 fragment, respectively. Reporter A was observed at high intensities for all analytes except homocysteine, indicating some structural dependence of this fragmentation scheme. Reporter B was used for quantitation of this analyte instead.

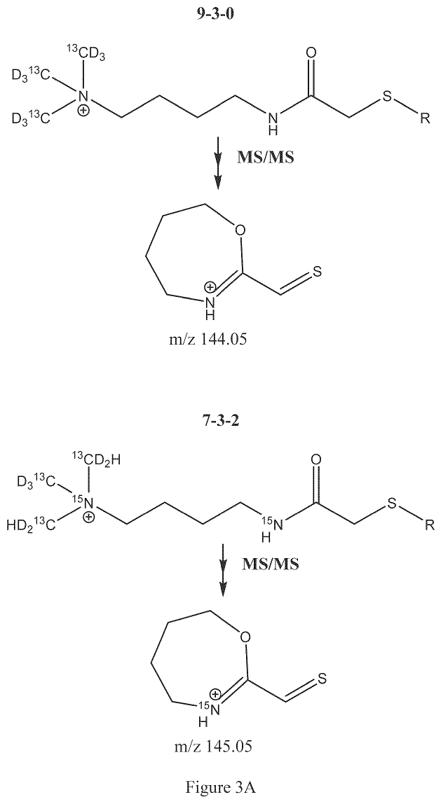

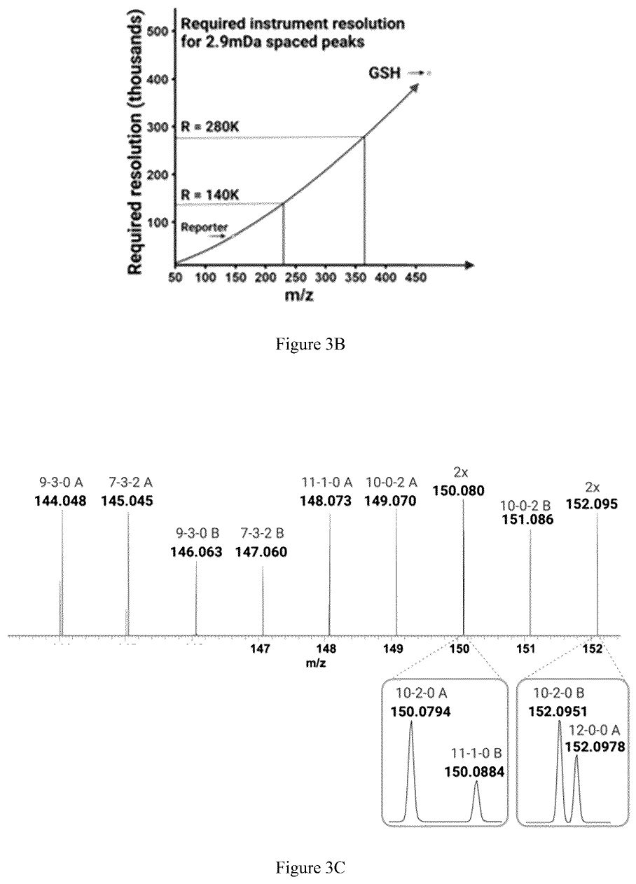

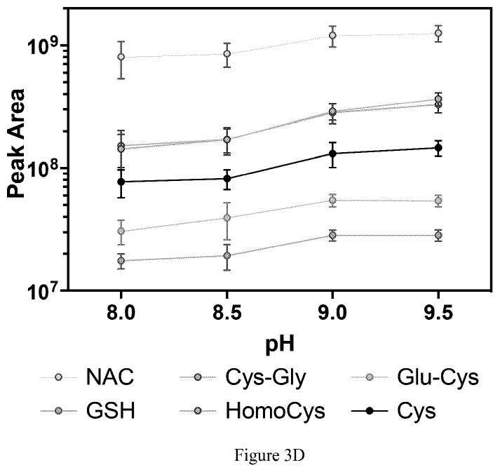

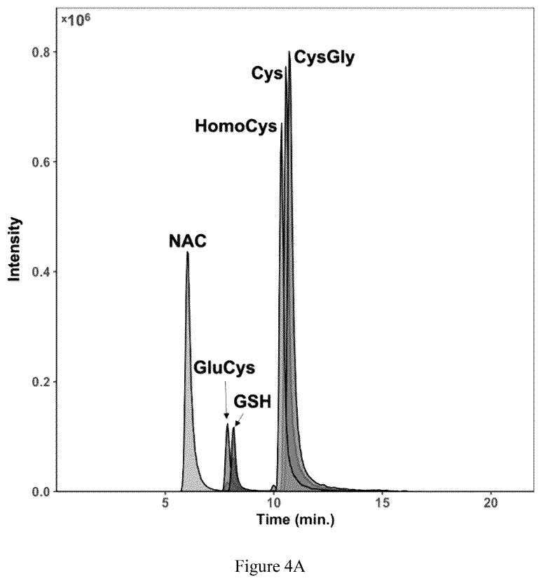

The secondary fragments produced by this tag are similar to traditional isobaric reporters due to their constant and low mass. This simplifies data analysis and provides improved resolution on orbitrap instruments without sacrificing MS² acquisition time (FIG. 3A-FIG. 3B). The resolution requirements for 2.9 mDa spaced peaks at a 10:1 ratio were calculated as previously described (Merrill, A. E., et al., Mol Cell Proteomics 2014, 13 (9), 2503-12). Although a pair of reporters spaced 2 Da apart are produced, there is no interference across isotope reporters due to the high resolution MS² scans (FIG. 3C). This allows for multiplexed analysis of higher mass metabolites like GSH without the extremely high resolution requirements of MS¹ based mass shift tags. Although a 6-plex is presented here, removing tag 10-2-0 would further reduce the resolution requirements by eliminating possible interference from the B reporters. Using this setup would produce reporters at m/z 144, 145, 148, 149, and 152 with their B reporters occupying empty lanes. A 6-plex was chosen here as it is amenable to triplicate analysis of two conditions in a single injection. The isotope encoded reporter m/z for each tag is shown below in Table 3.

TABLE 3
Isotope encoded reporter m/z.

| D | ¹³C | ¹⁵N | Reporter m/z |
|---|---|---|---|
| 9 | 3 | 0 | 144.0478 |
| 7 | 3 | 2 | 145.0448 |
| 11 | 1 | 0 | 148.0729 |
| 10 | 0 | 2 | 149.0699 |
| 10 | 2 | 0 | 150.0796 |
| 12 | 0 | 0 | 152.0980 |

Previous coupling of alkyl iodide tags to free thiols has used slightly alkaline conditions for coupling. An optimal pH allows for deprotonation of the free thiol group without deprotonating and activating amine groups. The pKa of thiol groups are influenced by nearby moieties and thus the reaction pH was optimized for the group of targeted analytes. In line with previous studies, increased reaction efficiency is observed at pH 9 (FIG. 3D) for samples reacted overnight.

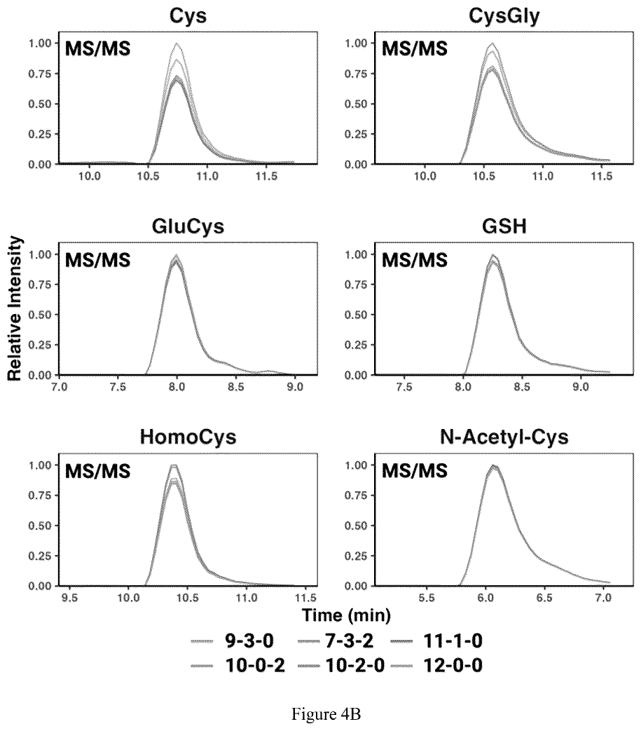

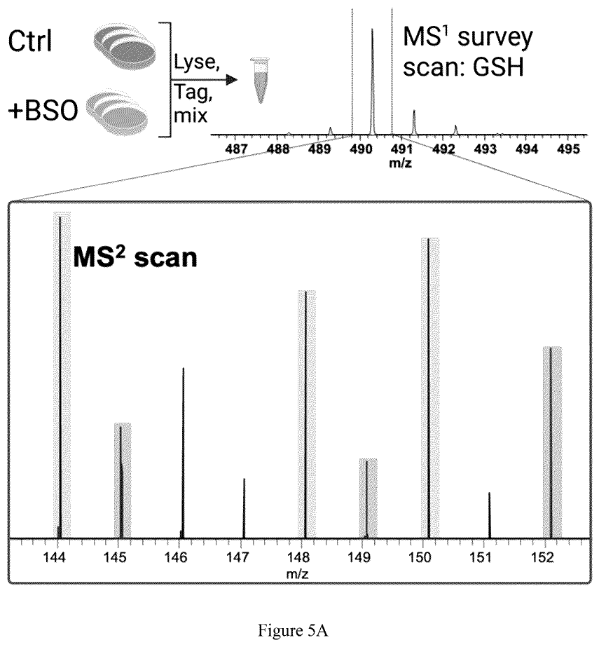

The six targeted thiols were separated using a HILIC method to leverage the high native polarity of the metabolites in addition to the quaternary amine moiety of the tag (FIG. 4A). It has previously been shown that incorporation of deuterium around a polar center mitigated retention time shifts for reverse phase separations. Here a similar trend is observed, as tagged thiols with a total deuterium content from 7 to 12 co-elute (FIG. 4B). This produces consistent reporter ratios across the peak, improving quantitation and reproducibility.

A major advantage of neutral loss-based tags is the quaternary amine group which provides a fixed positive charge and many sites of isotope incorporation. Isotope availability on the balancer group often limits multiplexing options on constant-mass reporter tags, as seen with the incorporation of an additional beta-alanine to the TMTpro balancer region. This larger tag now produces a total of 9 sites for ¹³C or ¹⁵N incorporation. Deuterium incorporation into these tags is currently unavailable due to concerns about chromatograph shifts, especially for small analytes. By negating deuterium shifts up to 13 isotopes are able to be incorporated on a small balancer region and incorporate a third type of isotope (¹³C, D, and ¹⁵N) which will enable higher levels of multiplexing.

Reaction reproducibility was assessed by mixing 6-plex tagged thiols 1:1 (n=6), producing an average RSD of 13.9% (Table 4). The addition of a quaternary amine group improves signal response by eliminating the need for protonation during electrospray. Signal to noise was improved by a factor of 10 upon tagging while maintaining linearity with an average R² of 0.98 across all 6 analytes at mixing ratios of 1:2:5:10 from 500 nM to 5 μM (Table 4).

TABLE 4
System performance and reproducibility.

| | | | | RT RSD | |
|---|---|---|---|---|---|
| | Intensity | | | (intra- | Optimal |
| | RSD | R² | RT | injection, | nCID |
| Analyte | (n = 6) | (n = 3) | (minutes) | n = 6) | (eV) |
| Cys | 17.1% | 0.972 | 10.71 | 0.31% | 50 |
| Homocysteine | 20.7% | 0.981 | 10.39 | 0.16% | 45 |
| NAC | 4.0% | 0.988 | 6.08 | 0.32% | 55 |
| Cys-Gly | 10.8% | 0.987 | 10.58 | 0.05% | 50 |
| γ-Glu-Cys | 18.3% | 0.980 | 7.97 | 0.67% | 50 |
| GSH | 12.7% | 0.970 | 8.23 | 0.45% | 50 |
| Average | 13.9% | 0.980 | 8.99 | 0.33% | 50 |

RT = retention time, RSD = relative standard deviation, nCID = normalized collision induced dissociation energy.

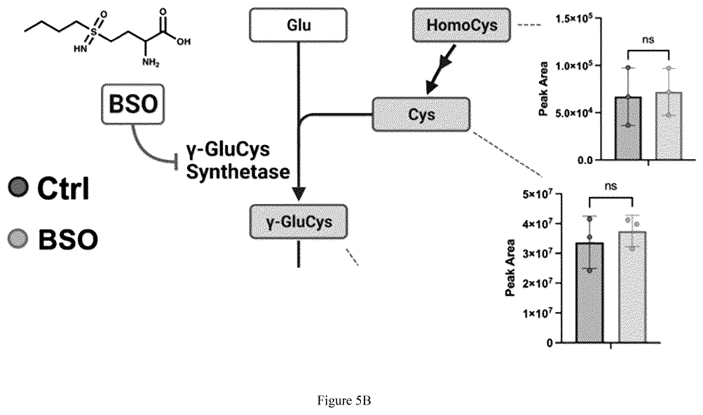

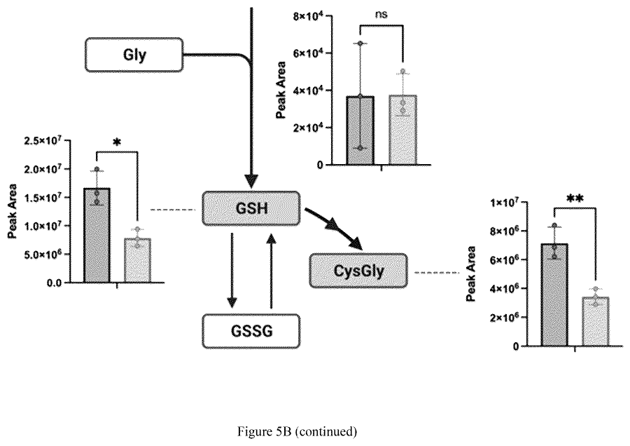

Buthionine sulfoximine (BSO) is an irreversible gamma glutamyl-cysteine synthetase inhibitor which decreases GSH stores. This was used to uncover thiol metabolism dysregulation in a complex biological system. Bovine aortic endothelial cells were treated with either reduced-serum media (2% fetal bovine serum in DMEM) or reduced serum media with 250 μM BSO for 16 hours (n=3). Cells were then lysed and tagged with the six isotope tag variants, mixed, dried, and injected (FIG. 5A).

Significant decreases were observed for GSH and its breakdown product cysteinyl-glycine, consistent with previous studies (FIG. 5B). Glutamyl-cysteine did not change significantly but was near the limit of detection which impacts analytical performance. While cysteine and homocysteine trended upward, they did not change. These two metabolites may be shunted toward other cysteine-containing antioxidants upon inhibition of GSH synthesis.

Here a 6-plex thiol metabolomics method was presented which used a unique double fragmentation route to produce constant-mass reporters. Each tag was synthesized using readily available isotope starting materials and simple synthetic reactions. As a proof-of-concept, glutathione depletion was observed upon treatment with BSO, which was consistent with previous studies. This analysis of 6 samples was completed in a single injection requiring 20 minutes of instrument time.

Isobaric tags most often rely on al type fragmentation to produce constant mass reporters. This limits the structural, and therefore synthetic options for tag creation. The use of double fragmentation creates opportunities for isobaric tags. The simple structure of the tag, the availability of inexpensive starting materials, and the ease of synthesis opens an arena for multiplexing in metabolomics. In addition, the use of bare silica chromatography maintains cost-efficient analysis. These advantages are shown by cost-effective isotope addition selective methylations, which allow for a fixed positive charge and additional methyl group compared to traditional tertiary amine reporters. It is anticipated that it is possible to transfer similar fragmentation mechanisms to different functional groups, allowing for broader coverage of the metabolome.

### Example 3: Expansion of Fragmentation Technique

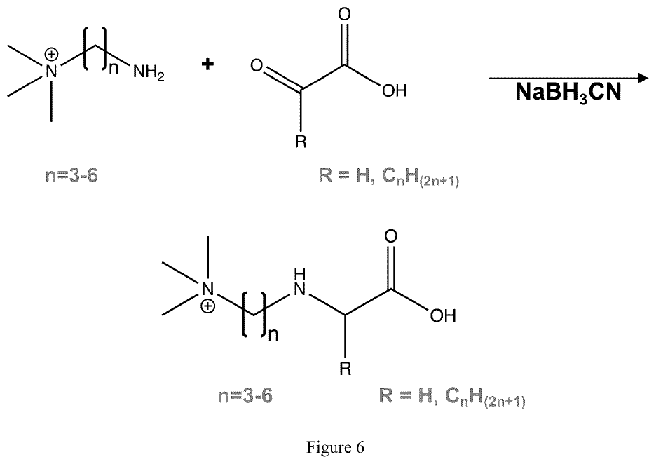

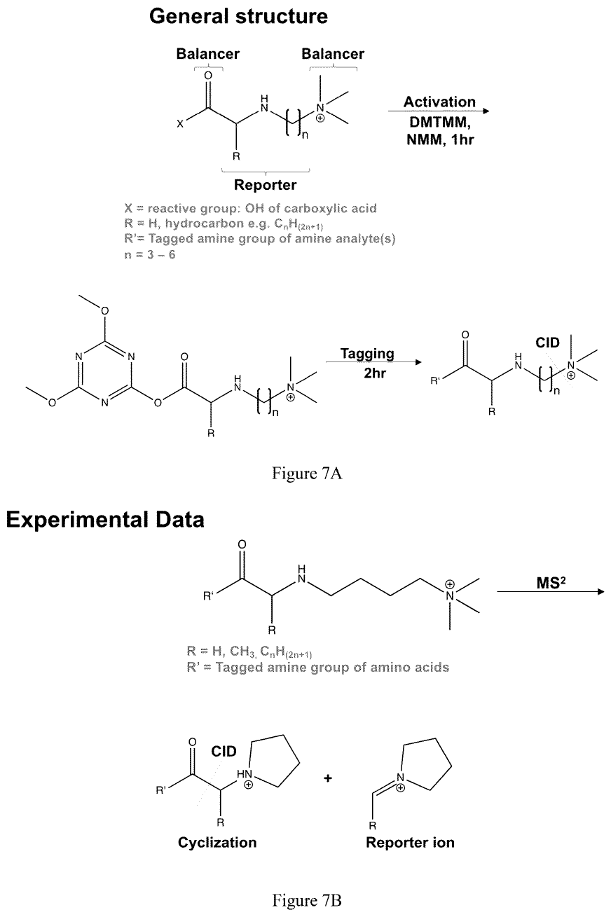

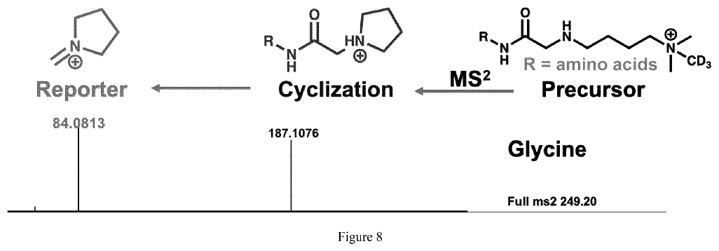

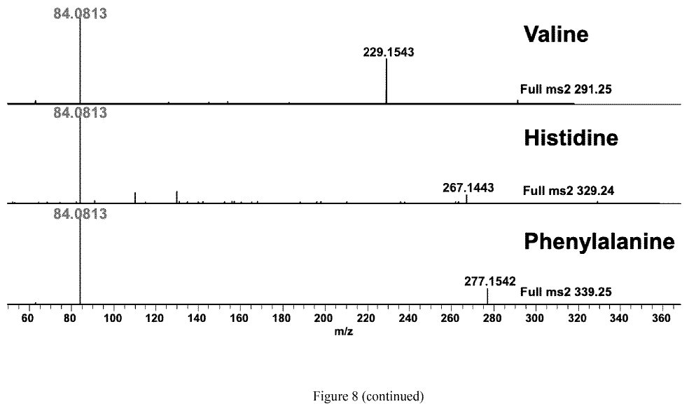

This dual fragmentation concept can be extended to mimic isobaric tags which rely on al type fragmentation. This is achieved by attaching the quaternary amine to alpha-keto acids through a reductive amination (FIG. 6). Here, the free acid acts as the reactive group which can be activated with DMTMM as previously described (Xiang, F., et al., Analytical Chemistry 2010, 82 (7), 2817-2825.) (FIG. 7A). These tags undergo similar cyclization upon collision induced dissociation (CID) to produce constant mass reporters (FIG. 7B). Glyoxylic acid represents the simplest tag, as R=H, and was used to investigate preliminary fragmentation efficiency. This tag variant was tested on amino acids and produced consistent and efficient reporters across a range of structures (FIG. 8). The quaternary amine tag used here contained a CD₃ group in the balancer region which is lost during fragmentation.

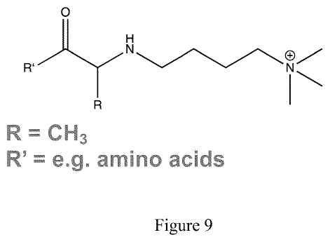

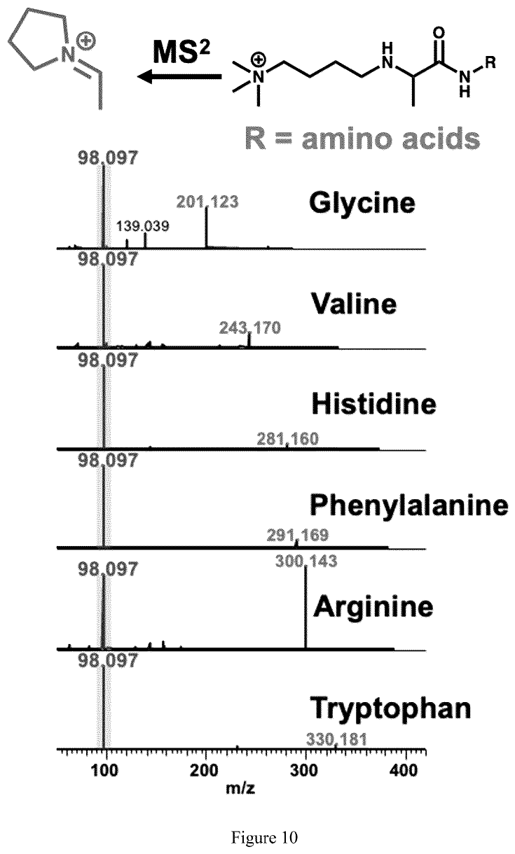

The R group on this tag can be extended to allow for more sites of isotope incorporation on the reporter. R group expansion was investigated using pyruvate (R=CH₃) as the linker (FIG. 9). This linker was chosen due to the many isotopically labeled variants commercially available. Again excellent fragmentation efficiency is observed with these tags despite the added bulk from the methyl group (FIG. 10).

Table 5 shows how commercially available isotopic variants of pyruvate allow for simple isotope incorporation. Nominal isotope mass shifts are shown for the net, balancer, and reporter shift. All isotopes are ¹³C except for P3-0 which contains 3 deuterium atoms.

TABLE 5
Pyruvate isotopes.

| Abbreviated | | | |
|---|---|---|---|
| name (B-R) | Net Shift | Balancer | Reporter |
| P0-0 | 0 | 0 | 0 |
| P1-0 | 1 | 1 | 0 |
| P2-1 | 3 | 1 | 2 |
| P2-0 | 2 | 0 | 2 |
| P0-1 | 1 | 0 | 1 |
| P1-1 | 2 | 1 | 1 |
| P2-0 | 2 | 0 | 2 |
| P3-0 (Deuterated) | 3 | 0 | 3 |

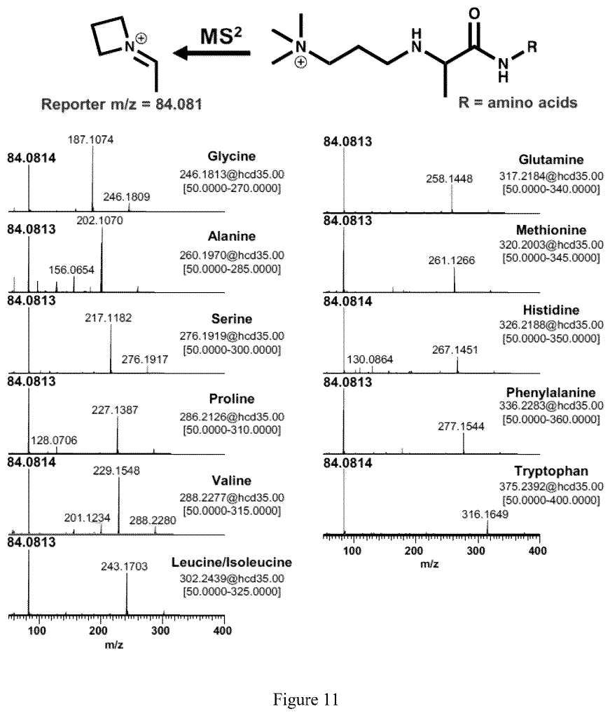

The chain length (n, FIG. 6) on the alpha-keto tags may be changed to further modify the reporter mass. The chain length was shortened to 3, which creates a 4 membered ring on the cyclized reporter (FIG. 11).

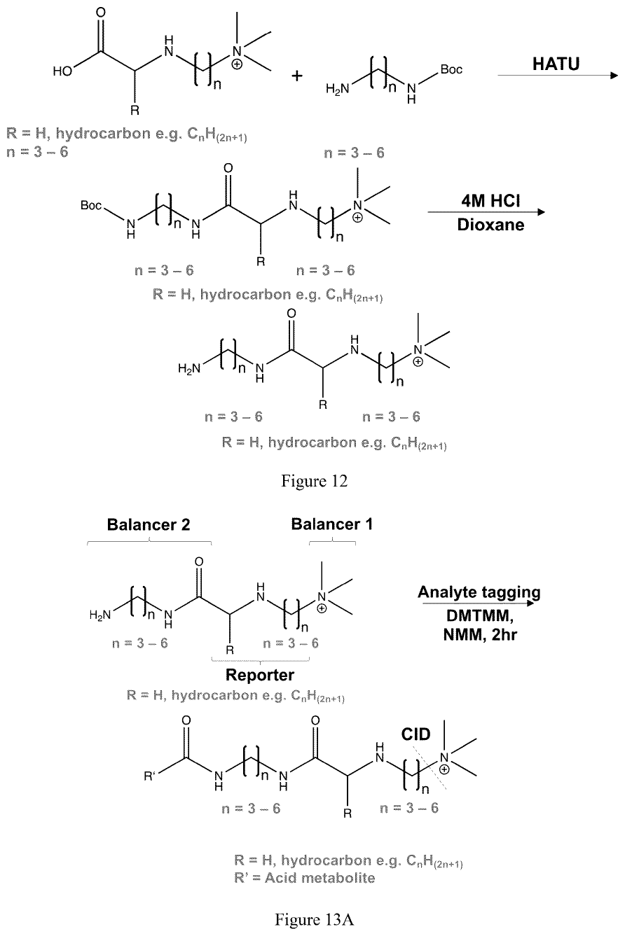

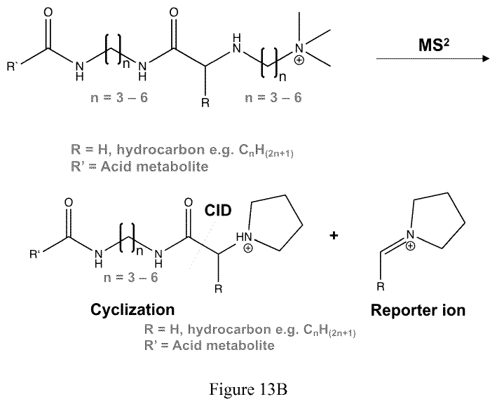

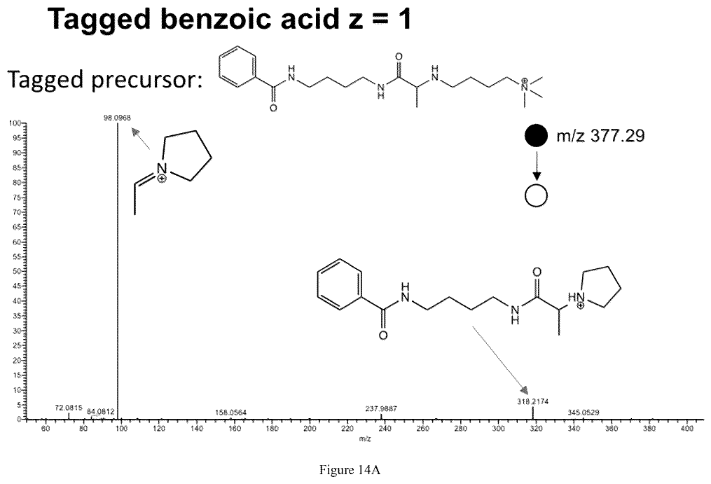

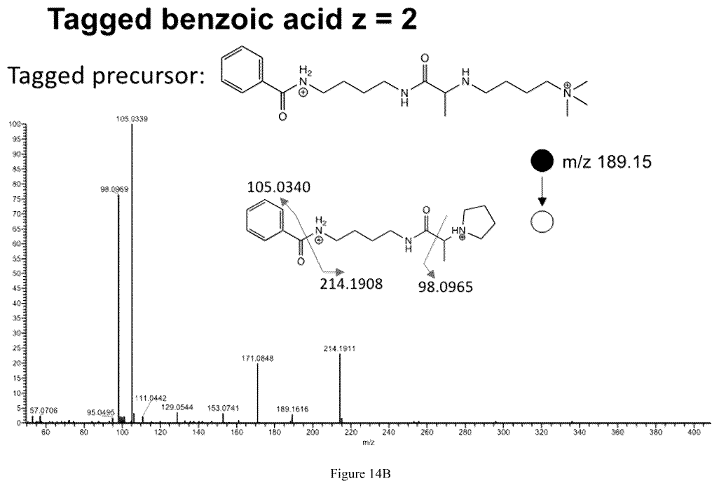

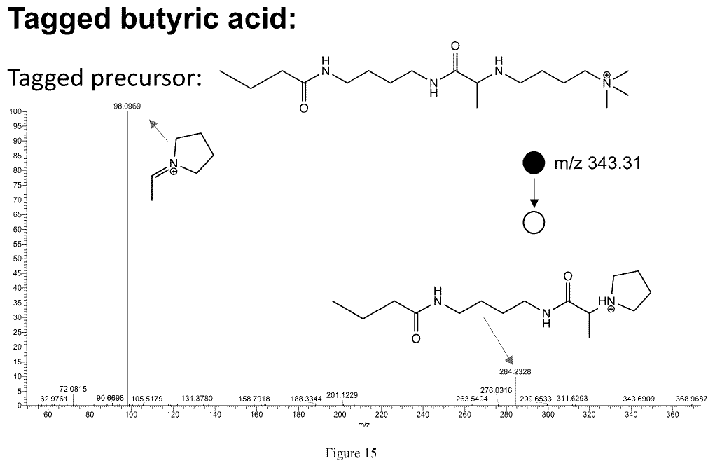

This alpha-keto tag was further modified to terminate with a primary amine (FIG. 12). A mono-boc protected alkyl diamine is coupled to the acid group on the alpha-keto tag, followed by deprotection with 4M HCl in dioxane. This allows for acid metabolite analysis through amide coupling and further isotope incorporation onto the balancer group (FIG. 13A). Like the previous variants, a trimethylamine loss and cyclization is followed by an al type fragmentation to produce constant mass reporter ions (FIG. 13B). The pyruvate tag variant (R=CH₃) was used for preliminary tests on benzoic acid (FIG. 14A-FIG. 14B) and butyric acid (FIG. 15). While tagged butyric acid was singly charged, tagged benzoic acid produced both singly and double charged variants. Fragmentation of these products consistently produced the expected cyclized reporter across charge states and structures.

What is claimed is:

1. An isobaric, double fragmentation mass spectrometry tag, wherein the tag is selected from the group consisting of:
a thiol tag of structure 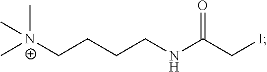
an amine tag of structure 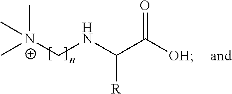
an acid and aldehyde tag of structure 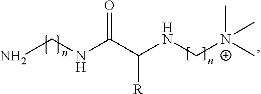
wherein R is H or unsubstituted alkyl;
wherein n is 3 to 6; and
wherein the tag has at least one N replaced by ¹⁵N, at least one C replaced by ¹³C,
and/or at least one H replaced by deuterium (D).

2. The tag of claim 1, wherein R is CH₃.

3. The tag of claim 1, wherein R is H.

4. The tag of claim 1, wherein n is 4.

5. The tag of claim 1, wherein at least 12 total N, C, and H atoms are replaced by ¹⁵N, ¹³C, and D, respectively.

6. The tag of claim 1, wherein the tag is selected from the group consisting of

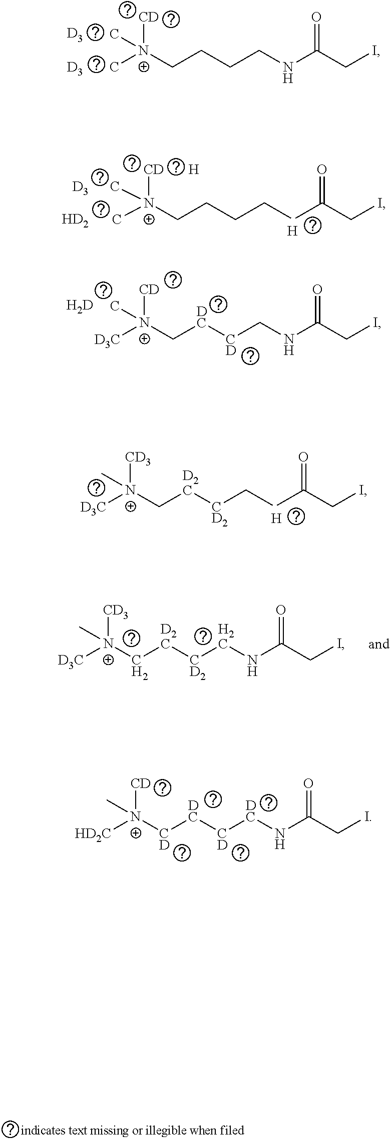

7. A method of multiplexed mass spectrometry for quantifying one or more analytes of interest in two or more samples, wherein the method comprises the following steps:
A) each sample is tagged with a different tag of claim 1, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight;
B) the samples are combined and processed via liquid chromatography;
C) a low resolution MS¹ scan is performed to identify the one or more analytes of interest and the tagged one or more analytes are fragmented to cyclize; and
D) secondary fragments of the tags are produced after a second fragmentation event and a high resolution MS² scan is performed to quantify the relative amount of the one or more analytes in each sample.

8. The method of claim 7, wherein each sample comprises lysed cells.

9. The method of claim 7, wherein each sample is derived from a bodily fluid or body tissue.

10. The method of claim 7, wherein the method has six samples.

11. The method of claim 10, wherein each tag is selected from the group consisting of

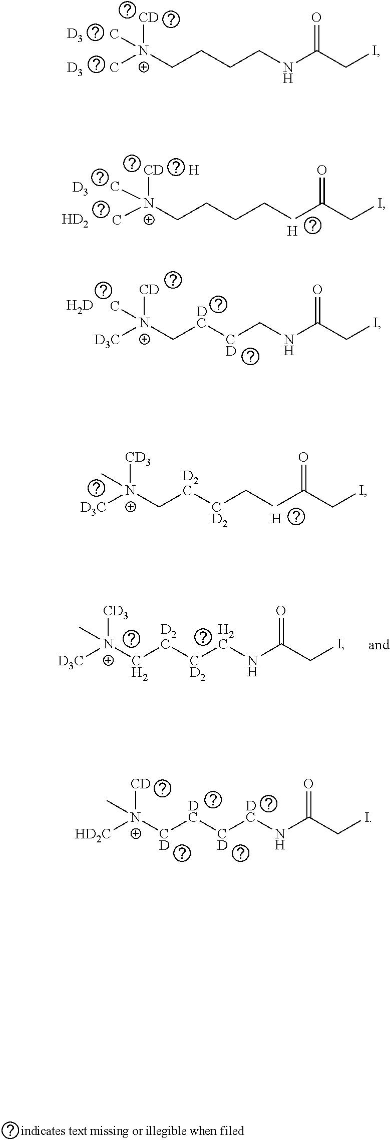

12. The method of claim 7, wherein the method has up to 192 samples.

13. The method of claim 7, wherein the one or more analytes comprise thiol-containing metabolites related to GSH metabolism.

14. The method of claim 7, wherein the one or more analytes is selected from the list consisting of Cys, Homocysteine, NAC, Cys-Gly, γ-Glu-Cys, GSH, and combinations thereof.

15. The method of claim 7, wherein the one or more analytes is one or more amino acids.

16. The method of claim 7, wherein the liquid chromatography is bare silica chromatography.

17. A kit for tagging two or more samples for multiplexed mass spectrometry of one or more analytes of interest, the kit comprising at least two of the isobaric, double fragmentation mass spectrometry tags of claim 1, wherein the kit comprises one tag for each sample, wherein all tags are a thiol tag if the one or more analytes is tagged via a thiol, all tags are an amine tag if the one or more analytes is tagged via an amine, and all tags are an acid and aldehyde tag if the one or more analytes is tagged via an acid or aldehyde; wherein each tag has the same nominal molecular weight.

18. The kit of claim 17, wherein the kit comprises at least six tags.

19. The kit of claim 17, wherein each tag is selected from the group consisting of

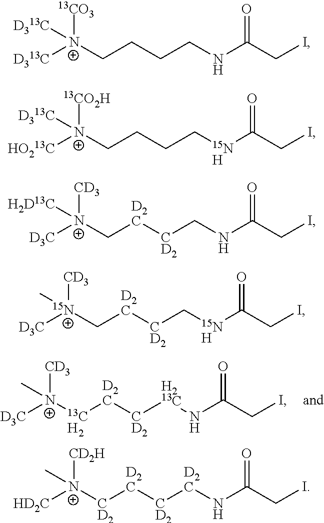

20. The kit of claim 17, wherein the kit comprises up to 192 tags.
